(19) United States
(12) Patent Application Publication
(10) Pub. No.: US 2018/0021145 A1
(43) Pub. Date: Jan. 25, 2018

(54) PROSTHETIC COMPONENT AND ASSOCIATED METHODS FOR A BONE FUSION PROCEDURE

(71) Applicant: Restore Surgical LLC. dba Instratek, Houston, TX (US)

(72) Inventors: Jeffrey F. Seavey, Houston, TX (US); Lance N. Terrill, League City, TX (US); Adam B. Trump, Farmington, UT (US); Kasey A. Kilgore, Houston, TX (US)

(21) Appl. No.: 15/654,055

(22) Filed: Jul. 19, 2017

Related U.S. Application Data
(60) Provisional application No. 62/364,816, filed on Jul. 20, 2016.

(51) Int. Cl. A61F 2/42; A61F 2/30

(52) U.S. Cl. CPC A61F 2/4225; A61F 2/30771; A61F 2002/4233; A61F 2002/30382; A61F 2002/30878; A61F 2002/30622

(57) ABSTRACT

A prosthetic component for use in a metatarso-phalangeal joint fusion procedure for a patient that includes a single monolithic body is disclosed. The body includes a distal body section that is sized to be positioned in a surgically-prepared bore defined in a proximal phalange of the patient's foot, and a proximal body section that is configured to be threaded into a surgically-prepared bore defined in a distal end of a metatarsal bone of the patient's foot. When the single monolithic body is viewed in a first plane, the distal body section may be laterally offset from the proximal body section when the single monolithic body is viewed in the first plane. A method of use and a method of removal are also disclosed.

This application claims priority to U.S. Patent App. Ser. No. 62/364,816, which was filed on Jul. 20, 2016 and is incorporated herein by reference.

### TECHNICAL FIELD

The present disclosure relates generally to prosthetic devices and, more specifically, to prosthetic implants for use in a metatarso-phalangeal joint of a patient.

### BACKGROUND

Repair of joints often involves the use of fixation devices to secure parts of the bony anatomy together during the healing process. There are many forms of bone fixation devices, including intramedullary devices, pins, screws, plates, and staples. In some cases, it may be necessary to fuse two bones together permanently. One such procedure is a metatarso-phalangeal joint (MPJ) fusion procedure.

Various methods and implants have been developed for use in an MPJ fusion procedure. Methods and implants are described in U.S. Pat. Nos. 8,920,476; 8,920,453; and 9,017,329. This listing is not intended as a representation that a complete search of all relevant prior art has been conducted, or that no better references than those listed exist.

### SUMMARY

According to one aspect, a prosthetic component for use in a metatarso-phalangeal joint fusion procedure for a patient is disclosed. The prosthetic component comprises a single monolithic body including a first end that is sized to be positioned in a surgically-prepared bore defined in a proximal phalange of the patient's foot and a second end sized to be positioned in a surgically-prepared bore defined in a distal end of a metatarsal bone of the patient's foot. The first end, which may be a distal end, is laterally offset from the second end when the single monolithic body is viewed in a first anatomical plane, such as, for example, the transverse plane of the patient's body.

In some embodiments, the second end of the single monolithic body may include a plurality of threads configured to be threaded into the surgically-prepared bore of the distal end of the metatarsal bone of the patient's foot.

In some embodiments, a first generally central axis may extend through the first end of the single monolithic body, a second generally central axis may extend through the second end of the single monolithic body, and an angle may be defined between the first generally central axis and the second generally central axis when the single monolithic body is viewed in the first plane. The angle may have a magnitude greater than 0 degrees. It should be appreciated that the angle may correspond to a desired valgus angle of the phalange relative to the metatarsal bone.

In some embodiments, the magnitude of the angle may be less than or equal to 20 degrees. In some embodiments, the magnitude may be equal to one of approximately 5 degrees, 10 degrees, and 20 degrees.

Additionally, in some embodiments, a second angle may be defined between the first generally central axis and the second generally central axis when the single monolithic body is viewed in a second anatomical plane extending orthogonal to the first plane. The second plane may be a sagittal plane of the patient's body, and the second angle may correspond to a desired dorsiflexion angle of the phalange relative to the metatarsal bone. The second angle may have a magnitude greater than 0 degrees.

In some embodiments, the magnitude of the second angle may be less than or equal to 35 degrees. In some embodiments, the magnitude of the second angle may be equal to approximately 20 degrees. As described above, in some embodiments, the first plane may be a transverse plane, and the second plane may be a sagittal plane.

In some embodiments, the single monolithic body may include a first body section that includes the first end and extends along the first generally central axis, a second body section that includes the second end and extends along the second generally central axis, and a central body section extending along a third generally central axis from a first section end connected to the first body section to a second section end connected to the second body section. A third angle may be defined between the first generally central axis and the third generally central axis when the single monolithic body is viewed in the first plane. The third angle may have a magnitude greater than 0 degrees.

In some embodiments, a spline may extend along the first end of the single monolithic body. The spline may be configured to engage bone surrounding the surgically-prepared bore defined in the proximal phalange of the patient's foot. In some embodiments, the spline may be one of a plurality of splines arranged around an outer circumference of the first end of the single monolithic body.

Additionally, in some embodiments, the single monolithic body may have a passageway defined therein that is sized to receive a fastener. In some embodiments, the passageway may be threaded at least one end.

In some embodiments, a surgical kit may comprise the prosthetic component described herein and further comprise a surgical tool including an offset corresponding to the offset of the first end relative to the second end and Kirschner wire.

According to another aspect, a prosthetic component for use in a metatarso-phalangeal joint fusion procedure for a patient comprises a single monolithic body. The body includes a distal body section that is sized to be positioned in a surgically-prepared bore defined in a proximal phalange of the patient's foot, and a proximal body section that is configured to be threaded into a surgically-prepared bore defined in a distal end of a metatarsal bone of the patient's foot. When the single monolithic body is viewed in a first plane, an angle is defined between a first generally central axis extending along the distal body section and a second generally central axis extending along the proximal body section. The angle has a magnitude greater than 0 degrees. Additionally, the distal body section is laterally offset from the proximal body section when the single monolithic body is viewed in the first plane.

In some embodiments, a second angle may be defined between the first generally central axis and the second generally central axis when the single monolithic body is viewed in a second plane extending orthogonal to the first plane. The second angle may have a magnitude greater than 0 degrees.

In some embodiments, a generally central spline may extend along the distal body section, and the spline may be configured to engage bone surrounding the surgically-prepared bore defined in the proximal phalange of the patient's foot. In some embodiments, the spline may be one of a plurality of splines arranged around an outer circumference of the distal body section.

According to another aspect, a method of performing a procedure on a patient is disclosed. The method comprises reaming a proximal phalange of the patient's foot, reaming a distal end of a metatarsal bone of the patient's foot, selecting a prosthetic component including a first end and a plurality of threads on a second end opposite the first end, threading the second end of the prosthetic component into the distal end of a metatarsal bone of the patient's foot, inserting a fastener through a passageway defined in the prosthetic component, and engaging the first end of the prosthetic component with the proximal phalange of the patient's foot after inserting the fastener. In some embodiments, the fastener may include at least one of a Kirschner wire and a screw.

In some embodiments, the method may also comprise driving a Kirschner wire into the proximal phalange after engaging the first end of the prosthetic component with the proximal phalange of the patient's foot. In some embodiments, advancing a screw into the surgically-prepared opening may include advancing the screw along the Kirschner wire. In some embodiments, the method may further comprise angling the proximal phalange relative to the distal end of the metatarsal bone before engaging the first end of the prosthetic component with the proximal phalange.

In some embodiments, engaging the first end of the prosthetic component with the proximal phalange of the patient's foot may include engaging at least one spline formed on the first end of the prosthetic component with the proximal phalange of the patient's foot.

According to another aspect, a method of performing a procedure on a patient comprises reaming a proximal phalange of the patient's foot, reaming a distal end of a metatarsal bone of the patient's foot, selecting a prosthetic component a first end and a plurality of threads on a second end opposite the first end, threading the second end of the prosthetic component into the distal end of a metatarsal bone of the patient's foot, inserting a Kirschner wire through a passageway defined in the prosthetic component, engaging the first end of the prosthetic component with the proximal phalange of the patient's foot after inserting the Kirschner wire, driving the Kirschner wire into the proximal phalange to define a surgically-prepared opening in the proximal phalange, removing the Kirschner wire after driving the Kirschner wire into the proximal phalange, and advancing a screw into the surgically-prepared opening.

In other embodiments, advancing the screw into the surgically-prepared opening may include advancing the screw into the surgically-prepared opening after removing the Kirschner wire.

In some embodiments, preparing the proximal phalange of the patient's foot to receive a prosthetic component may include resecting the proximal phalange with a convex cup reamer having a first diameter, and preparing the distal end of the metatarsal bone of the patient's foot to receive the prosthetic component may include resecting the distal end with a concave cup reamer having a second diameter that is different from the first diameter. In some embodiments, the second diameter is less than the first diameter.

In some embodiments, engaging the first end of the prosthetic component with the proximal phalange of the patient's foot may include engaging at least one spline formed on the first end of the prosthetic component with the proximal phalange of the patient's foot.

According to another aspect, a method of removing a prosthetic component from a patient is disclosed. The method includes cutting the component to separate a distal body section positioned in a proximal phalange of the patient's foot from a proximal body section position in a distal end of a metatarsal bone of the patient's foot, inserting a threaded shaft into the distal body section, and pulling the threaded shaft away from the proximal phalange to withdraw the distal body section from the proximal phalange.

In some embodiments, the method may also include inserting a threaded shaft into the proximal body section to withdraw the proximal body section from the distal end of the patient's metatarsal bone.

### BRIEF DESCRIPTION OF THE DRAWINGS

The detailed description particularly refers to the following figures, in which:

FIG. 1 is a plan view of a prosthetic component for use in a metatarso-phalangeal joint fusion procedure;

FIG. 2 is another plan view of the prosthetic component of FIG. 1;

FIG. 3 is a side elevation view of the prosthetic component of FIG. 1;

FIG. 4 is a cross-sectional plan view of the prosthetic component of FIG. 1 implanted in a patient's metatarso-phalangeal joint;

FIG. 5 is a cross-sectional side elevation view of the prosthetic component of FIG. 1 implanted in a patient's metatarso-phalangeal joint;

FIG. 6 is a flow diagram of an implantation procedure for implanting the prosthetic component of FIG. 1;

FIGS. 7A-7B illustrate the bones of a patient's metatarso-phalangeal joint during the implantation procedure;

FIG. 8 shows a convex cup reamer for use during the implantation procedure;

FIG. 9 shows a concave cup reamer for use during the implantation procedure;

FIG. 10 shows a surgical trial component inserted into the surgically-prepared phalange and the surgically-prepared distal end of the metatarsal bone of FIG. 7B;

FIG. 11 shows a surgical instrument for inserting the prosthetic component of FIG. 1 coupled to one end of the prosthetic component;

FIG. 12 shows the prosthetic component of FIG. 1 positioned in the surgically prepared distal end of a metatarsal bone;

FIG. 13 is a view similar to FIG. 12 showing another step of the implantation procedure of FIG. 6;

FIG. 14 is a view similar to FIGS. 12-13 showing another step of the implantation procedure of FIG. 6;

FIG. 15 is a view similar to FIGS. 12-14 showing an optional step of the implantation procedure of FIG. 6; and

FIG. 16 is a flow diagram of a removal or revision procedure for removing the prosthetic component of FIG. 1 from a patient's metatarso-phalangeal joint.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

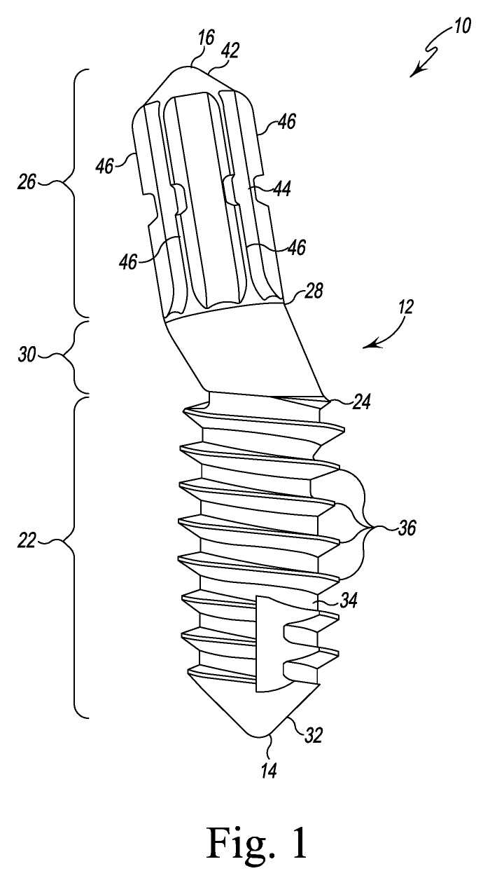

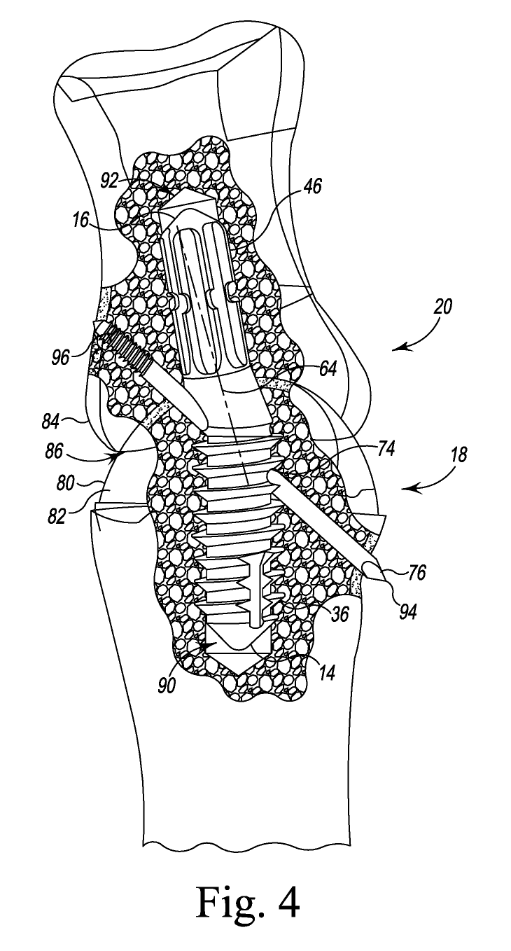

Referring now to FIG. 1, a prosthetic component 10 for use in a metatarso-phalangeal joint fusion procedure for a patient's toe is shown. The component 10 includes a single monolithic elongated body 12 that has a proximal end 14 to a distal end 16. As described in greater detail below, the proximal end 14 of the elongated body 12 is sized to be positioned in a surgically-prepared metatarsal bone 18 (see FIG. 4), while the distal end 16 is sized to be positioned in a surgically-prepared proximal phalange 20 (see FIG. 4) of a patient's foot. As shown in FIG. 1, the elongated body 12 does not extend along a single, straight line such that the distal end 16 is laterally offset from the proximal end 14 of the elongated body 12. Additionally, the elongated body 12 is shaped to generally match the patient's bony anatomy such that a predetermined valgus angle and a predetermined dorsiflexion angle are defined between the metatarsal bone 18 and the phalange 20, as described in greater detail below.

In the illustrative embodiment, the elongated body 12 is formed from an component-grade polymeric material such as, for example, polyether ether ketone (PEEK) that has been injection-molded into a single piece in the shape shown in FIG. 1. It should be appreciated that in other embodiments the elongated body may be formed from other suitable materials such as component-grade metallic materials. Examples of suitable component-grade metallic materials include titanium alloys. It should also be appreciated that the elongated body may be manufactured using other techniques such as, for example, machining.

The elongated body 12 may include a number of connected body sections between its ends 14, 16. For example, as shown in FIG. 1, a proximal body section 22 extends from the proximal end 14 to a proximal joint 24. The elongated body 12 also includes a distal body section 26 that extends from the distal end 16 to a distal joint 28. A central body section 30 extends between the proximal joint 24 and the distal joint 28. In the illustrative embodiment, the proximal body section 22 is configured to be threaded into the metatarsal bone 18 to secure the component 10 to the metatarsal bone. The distal body section 26 is sized and shaped to secure the component 10 to the phalange 20 via a press fit.

In the illustrative embodiment, the proximal body section 22 includes a tip 32 at the proximal end 14 and an outer surface 34 that extends between the tip 32 and the proximal joint 24. A plurality of threads 36 are arranged on the outer surface 34 such that the proximal body section 22 may be threaded into the distal end of the metatarsal bone 18. The threads 36 are illustratively self-tapping. In some embodiments, a threaded aperture may be defined in the proximal body section 22, which may be used with a removal tool to assist the surgeon in removing the proximal body section 22 from the patient's bone in a revision or like surgical procedure.

The distal body section 26 includes another tip 42 at the distal end 16 and a substantially cylindrical outer surface 44 that extends between the tip 42 and the distal joint 28. In the illustrative embodiment, the distal body section 26 is grit-blasted. In other embodiments, the body section 26 may be spray-coated with titanium plasma.

As shown in FIG. 1, the distal body section 26 has a plurality of splines 46 that extend outwardly from the cylindrical outer surface 44. The splines 46 are configured to engage the surgically-prepared phalange 20 to secure the distal body section 26 to the phalange 20. The splines 46 are illustrated as being arranged circumferentially around the outer surface 44. Illustratively, the splines 46 are equally spaced apart from one another. As described in greater detail below, at least one spline 46 is shorter in length than the other splines. It should be appreciated that in other embodiments the component 10 may include additional or fewer splines arranged in various patterns. In other embodiments, the component may include other fasteners configured to secure the component to the phalange. For example, spring-loaded arms, which may extend longitudinally, may be used in place of one or more of the splines 46. In other embodiments, the outer surface of the distal body section may be roughened or porous to grip the phalange. In still other embodiments, the distal body section may be tapered. Similar to the proximal body section, the distal body section may have a threaded aperture, which may be used with a removal tool to assist the surgeon in removing the distal body section from the patient's bone in a revision or like surgical procedure.

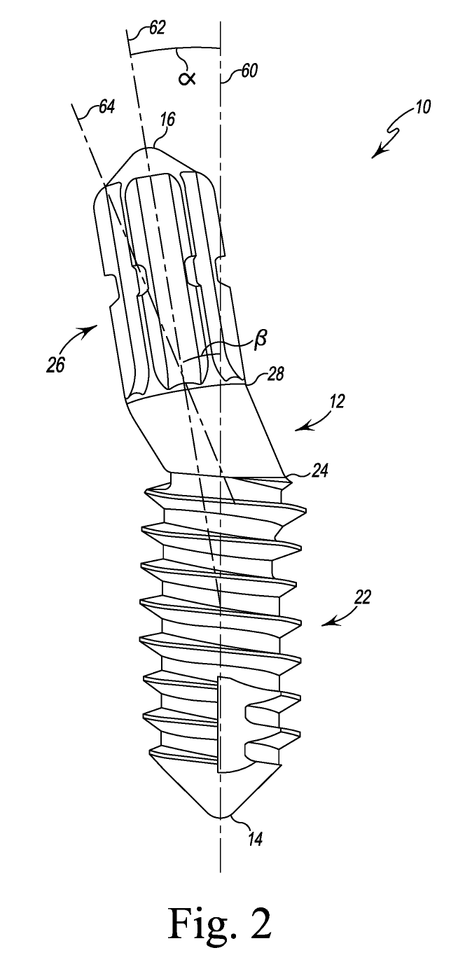

Referring now to FIG. 2, the proximal body section 22 extends along a central or generally central axis 60 that extends through the proximal end 14 of the component 10 and the proximal joint 24. The distal body section 26 extends along another central or generally central axis 62 extending through the distal end 16 of the component 10 and the distal joint 28. As shown in FIG. 2, the component 10 is arranged in a plane corresponding to the transverse plane of the patient's body. In that plane, an angle α is defined between the axes 60, 62. As described above, the distal end 16 is laterally offset from the proximal end 14. In some embodiments, the magnitude of the angle α may be 0 degrees; in other words, the axes 60, 62 may extend parallel to one another. In the illustrative embodiment, the angle α has a magnitude that is greater than 0 degrees such that a predetermined valgus angle is imparted when the component 10 is properly positioned in the patient's bones. In the illustrative embodiment, the magnitude of the angle α is less than or equal to 20 degrees. It should be appreciated that the angle α may also be equal to one of 5, 10, and 20 degrees.

As shown in FIG. 2, the central body section 30 extends along another central or generally central axis 64 extending through the joints 24, 28. In this illustrative embodiment, an angle β is defined between the axes 60, 64. In this embodiment, the angle β also has a magnitude greater than the magnitude of the angle α. As described in greater detail below, the central body section 30 is angled relative to the other body sections 22, 26 such that the axis 64 extends through the approximate radial centers of the surgically-prepared surfaces of the metatarsal bone 18 and the phalange 20 when the component 10 is properly positioned in the patient's body.

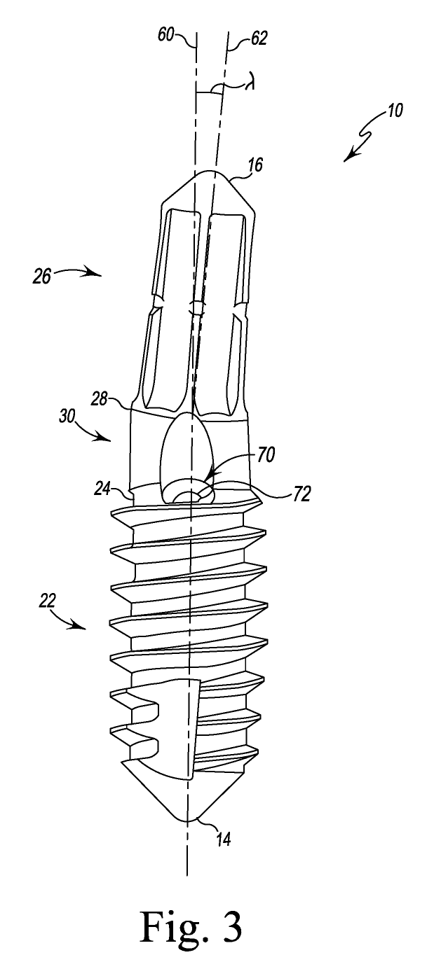

Referring now to FIG. 3, the component 10 includes a passageway 70 that extends through the elongated body 12. In the illustrative embodiment, the passageway 70 has an opening 72 defined in the central body section 30 and the joint 24 of the elongated body. The passageway 70 has another opening 74 (see FIG. 4) in the proximal body section 22 of the elongated body 12 such that the passageway 70 extends at an angle through the elongated body 12. As described in greater detail below, the passageway 70 is sized to receive a Kirschner wire 76 (see FIG. 4) and/or a bone screw 78 (see FIG. 15).

As shown in FIG. 3, the component 10 is arranged in a plane that is orthogonal to the plane shown in FIG. 2 and which may correspond to the sagittal plane of the patient's body. In that plane, an angle λ is defined between the axes 60, 62. In the illustrative embodiment, the angle λ has a magnitude that is greater than 0 degrees such that a predetermined dorsiflexion angle is imparted when the component 10 is properly positioned in the patient's bones. In the illustrative embodiment, the magnitude of the angle λ is less than or equal to 35 degrees such that a dorsiflexion angle of less than or equal to 20 degrees is imparted. It should be appreciated that the angle λ may be equal to 20 degrees. In some embodiments, the magnitude of the angle λ may be 0 degrees; in other words, the axes 60, 62 may be co incident with one another when viewed in the plane shown in FIG. 3. It should be appreciated that when the axes 60, 62 are positioned in a single plane in which all of the points of each axis lie in the plane, a compound angle having a magnitude between 5 and 16 degrees may be defined between the axes 60, 62.

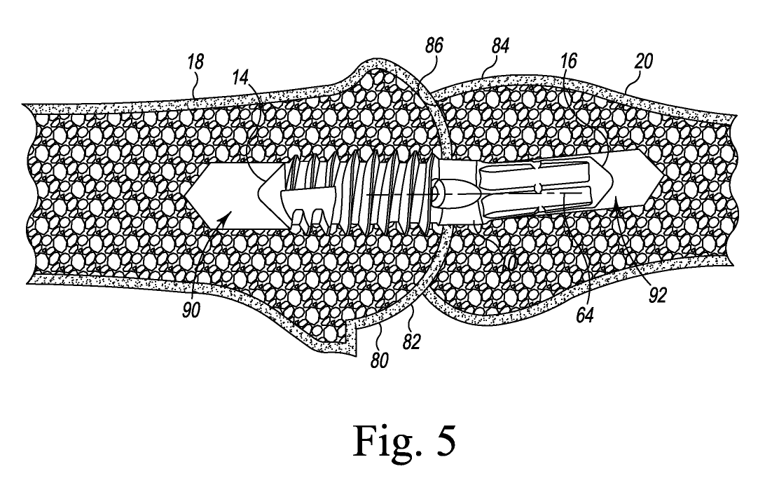

Referring now to FIGS. 4 and 5, the component 10 is shown positioned in a surgically-prepared metatarsal bone 18 and a surgically prepared phalange 20. In the illustrative embodiment, the distal end 80 of the metatarsal bone 18 has been resected to define a substantially hemispherical convex surface 82. The proximal end 84 of the phalange 20 has been resected to define a corresponding hemispherical concave surface 86 that is sized to receive the convex surface 82 of the bone 18. As described above, when the component 10 is positioned as shown in FIGS. 4-5, the axis 64 of the central body section 30 extends through the radial centers of the surfaces 82, 86 such that the surfaces are in full contact with one another.

The proximal body section 22 of the component 10 is positioned in a surgically-prepared bore 90 extending inwardly from the convex surface 82 of the metatarsal bone 18. Similarly, the distal body section 26 of the component 10 is positioned in a surgically prepared bore 92 extending inwardly from the concave surface 86 of the phalange 20. As shown in FIG. 4, a Kirschner wire 76 having a pointed proximal tip 94 and a threaded distal end 96 may be positioned in the passageway 70 of the component 10 to provide additional fixation. In the illustrative embodiment, the proximal tip 94 extends through the distal end 80 of the metatarsal bone 18, while the threaded distal end 96 of the wire 76 is positioned in the proximal end 84 of the phalange 20. As described in greater detail below, the wire 76 may remain implanted in the position shown in FIG. 4 at the conclusion of the surgical procedure; in other embodiments, a bone screw 78 may be implanted in place of the wire 76.

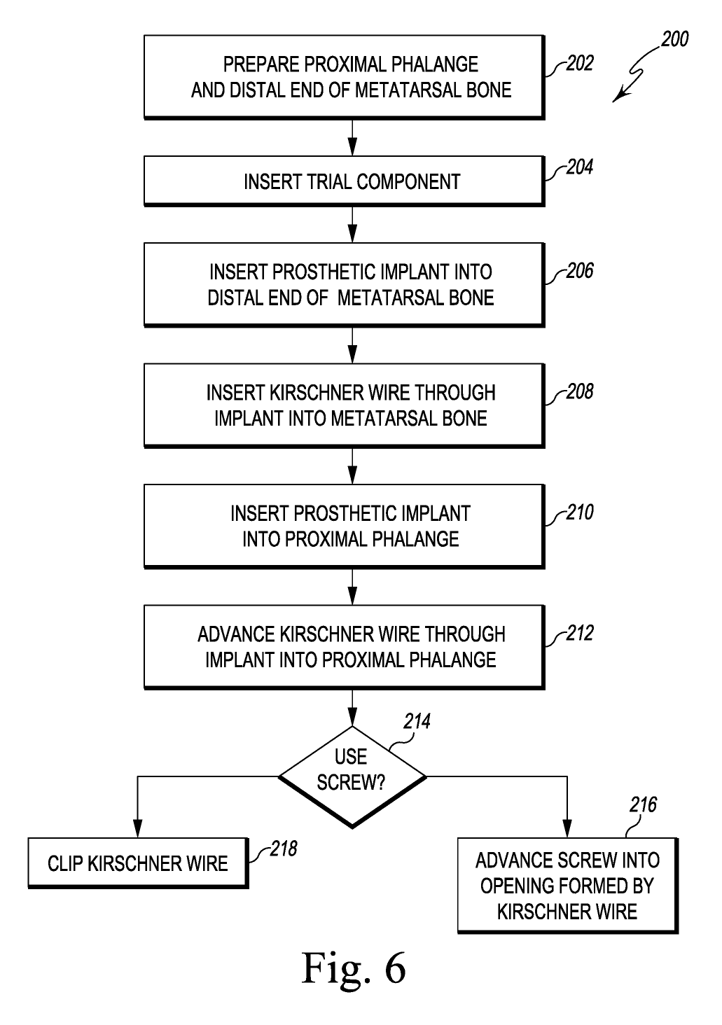

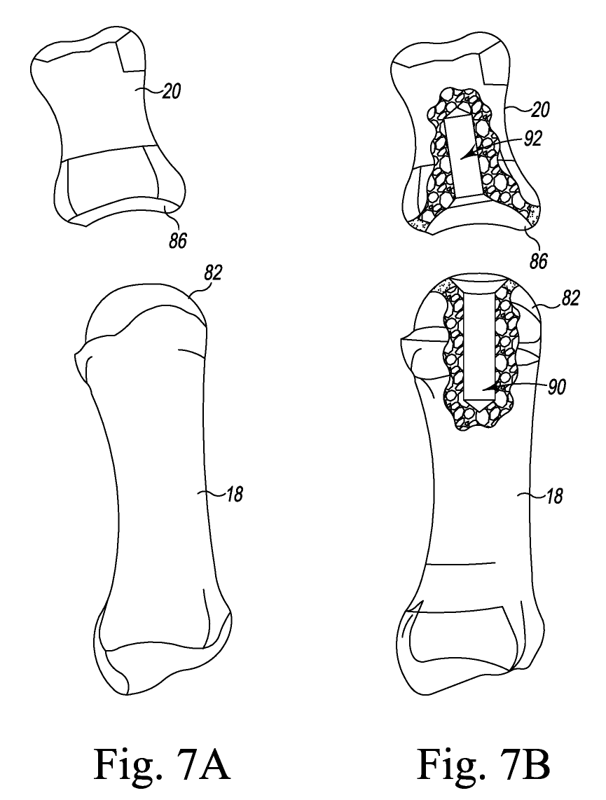

Referring now to FIG. 6, an exemplary method 200 for implanting the prosthetic component 10 is shown. The method 200 includes a number of surgical steps. It should be appreciated that in other embodiments some of the steps shown may be combined and/or omitted based on the needs of the patient. A number of steps of the method 200 are described below in reference to the illustration shown in FIGS. 7-15.

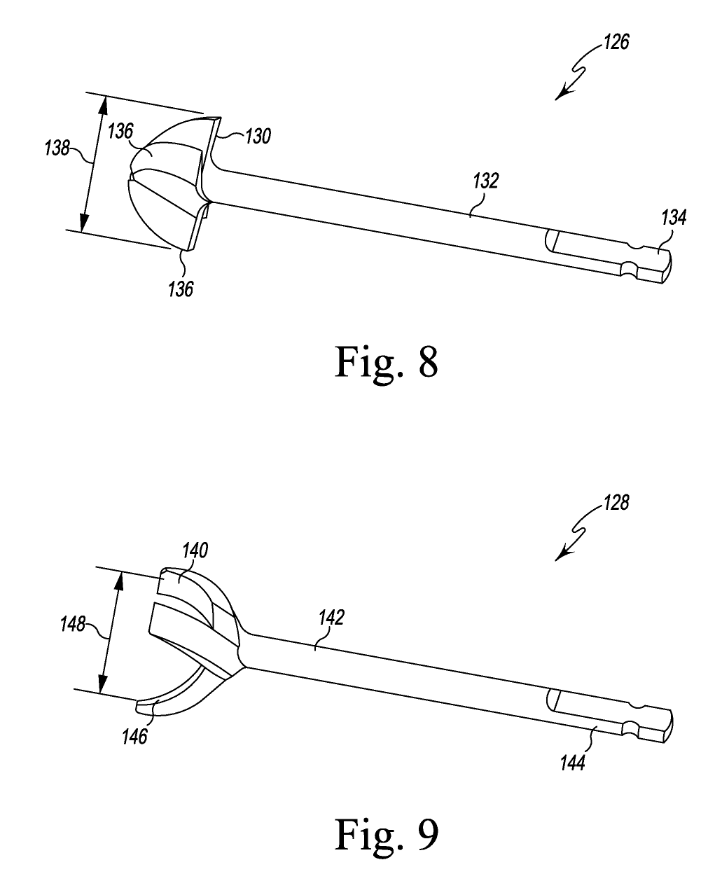

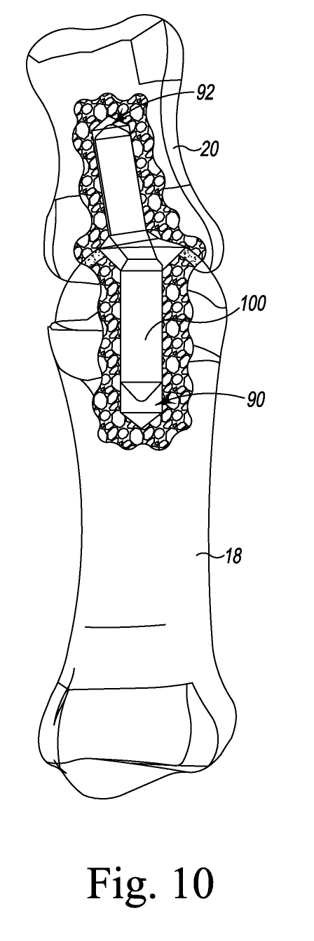

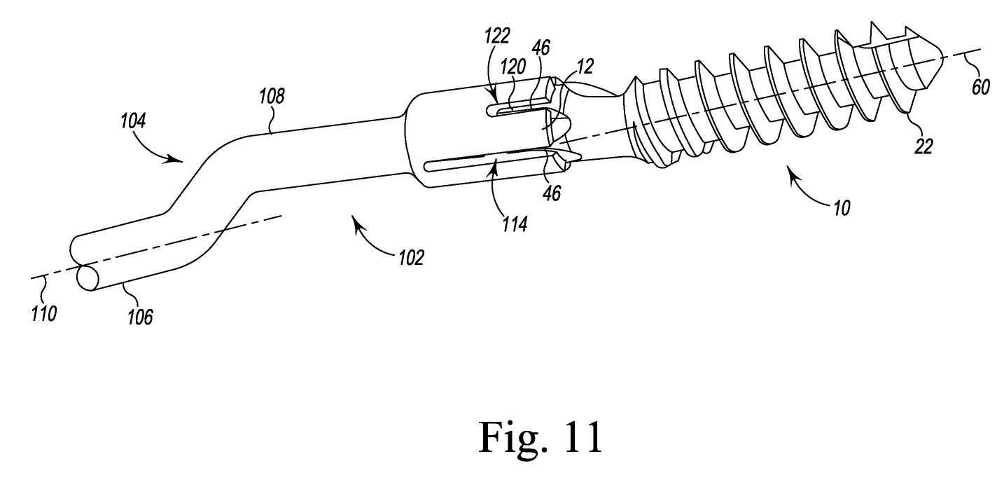

Prior to beginning the method 200, the surgeon or other medical staff may obtain one or more sterile surgical kits including the prosthetic component 10 and a number of surgical instruments, including, for example, Kirschner wire 76 and one or more bone cannulated screws 78. The kits may include one or more prosthetic components 10 of different sizes to be used with bones of different sizes. The kits may also include prosthetic components customized for use with a left foot or a right foot of a patient. The surgical instruments in the kit may include one or more trial component 100 (see FIG. 10), a driver 102 (see FIG. 11) configured to drive the prosthetic component 10 into the patient's bones, as described in greater detail below, and a number of spherical reamers (see FIGS. 8-9) configured to shape the patient's metatarsal bone 18 and the phalange 20 to receive the component 10.

The method 200 may begin with the surgeon opening the patient's tissue surrounding the metatarso-phalangeal joint to prepare the proximal phalange 20 and the metatarsal bone 18 in block 202. To prepare the proximal phalange 20, the surgeon may utilize a convex cup reamer 126 (see FIG. 8) to define the hemispherical concave surface 86 of the proximal phalange 20 that is shown in FIG. 7A. To prepare the distal end 80 of the metatarsal bone 18, the surgeon may utilize a corresponding concave cup reamer 128 (see FIG. 9) to define the hemispherical convex surface 82 of the distal end 80 of the metatarsal bone 18 that is shown in FIG. 7B. To define the bores 90, 92, a surgeon may utilize a surgical drill or reamer to create openings in the surfaces 82, 86 sized to receive the respective sections of the component 10, as shown in FIG. 7B.

As shown in FIG. 8, the convex cup reamer 126 includes a cutting head 130 and an elongated shaft 132 that extends from the cutting head 130 to a distal end 134 configured for attachment to a surgical drill or other surgical instrument. The cutting head 130 includes a plurality of cutting flutes 136 that define a substantially convex hemispherical shape. In the illustrative embodiment, the cutting head 130 has a cutting diameter 138 equal to 20 millimeters. It should be appreciated that the diameter may be increased or decreased depending on, for example, the patient's anatomy.

The corresponding concave cup reamer 128 is shown in FIG. 9. The reamer 128 includes a cutting head 140 and an elongated shaft 142 that extends from the cutting head 140 to a distal end 144 configured for attachment to a surgical drill or other surgical instrument. The cutting head 140 includes a plurality of cutting flutes 146 that define a substantially concave hemispherical shape. In the illustrative embodiment, the cutting head 140 has a cutting diameter 148 equal to 18 millimeters. It should be appreciated that the diameter may be increased or decreased depending on, for example, the patient's anatomy.

It should be appreciated that the cutting diameter of the concave reamer 128 is less than the cutting diameter of the convex reamer 126. This “mismatch” permits the shape and size of the prosthetic component 10 to define the alignment of the joint rather than permitting the center of rotation of the reamers to define the alignment. In the illustrative embodiment, the mismatch is equal to 2 millimeters, as measured on the diameter of the reamed surfaces.

After the reaming is complete, the method 200 may then advance to block 204 in which the surgeon may select a trial component 100 corresponding to the size and shape of a prosthetic component 10. The surgeon may then insert the trial component 100 into the bores 90, 92 defined in the bones 18, 20, as shown in FIG. 10. With the trial component 100 in place, the surgeon may move the bones to evaluate the preliminary fusion angle of the joint. Based on this evaluation, the surgeon may remove the first trial component 100 and select another trial component having a different size and shape. With the second trial component place, the surgeon may repeat the evaluation of the preliminary fusion angle. The surgeon may continue to repeat the process with different trial components until satisfied with the preliminary fusion angle. The surgeon may then select a prosthetic component 10 based on the final selected trial component. With the prosthetic component selected, the method may advance to block 206.

In block 206, the surgeon may attach the selected prosthetic component 10 to a driver 102, as shown in FIG. 11. The driver 102 includes an elongated body 104 having a drive shaft 106 and a mounting shaft 108 that is offset from the drive shaft 106. In the illustrative embodiment, the drive shaft 106 is operable to rotate about an axis 110 that is aligned with the axis 60 of the proximal body section of the component 10. The mounting shaft 108 has a distal end 112 that is configured to receive the distal body section 26 of the component 10. The distal end 112 has a number of slots 114 that extend from the distal end 112 and are sized to receive the splines 46 of the component 10. As described above, one of the splines 46, identified in FIG. 11 as a spline 120, is shorter in length than the other splines 46. One of the slots 114, identified in FIG. 11 as a slot 122, is similarly shorter in length than the other slots 114 and is sized to receive the spline 120. The combination of the spline 120 and slot 122 keys the component 10 into position relative to the driver 102, thereby preventing the surgeon from loading the component 10 in an improper orientation. As described above, in other embodiments the shorter spline may be omitted entirely to key the component 10 into position.

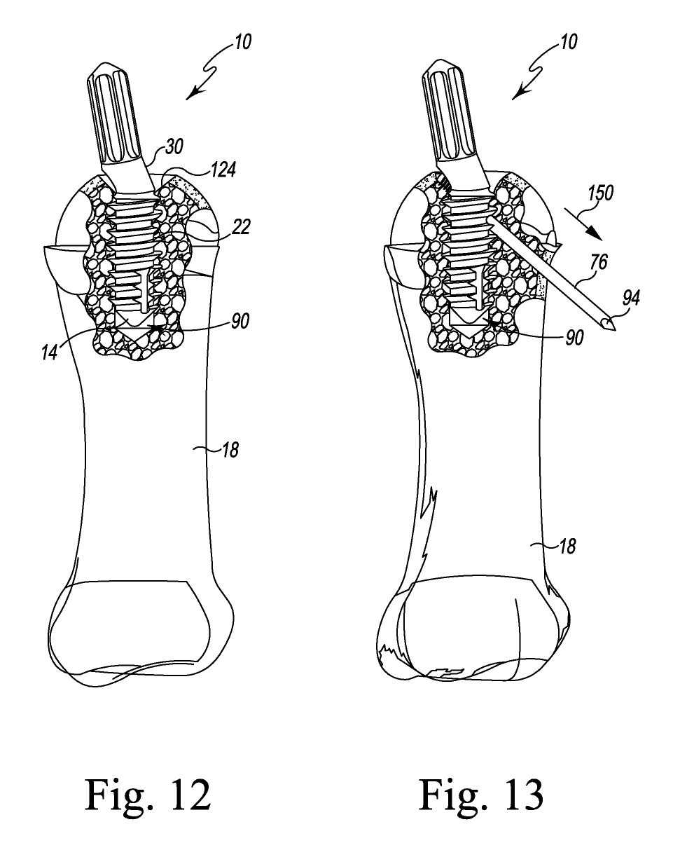

The surgeon may align the proximal end 14 of the component 10 with the bore 90 defined in the metatarsal bone 18 and then advance the proximal end 14 into the bore 90. When the threads 36 engage the bone surrounding the bore 90, the surgeon may rotate the driver 102 about its axis 110 to thread the proximal body section 22 into the bore 90. As shown in FIG. 12, when properly positioned in the bore 90, the proximal body section 22 is fully received in the bore 90. In the illustrative embodiment, the tilt or angle of the central body section 30 is accommodated in the bore 90 by an annular chamfer 124 defined at the end of the bore 90. The method 200 may then advance to block 208.

In block 208, the surgeon advances a Kirschner wire 76 through the prosthetic component 10 and the metatarsal bone 18, as shown in FIG. 13. To do so, the surgeon may align the wire 76 with the distal opening 72 of the passageway 70 defined in the component 10. The surgeon may then advance the pointed proximal tip 94 of the wire 76 into the distal opening 72, through the passageway 70, and into the metatarsal bone 18, as indicated by arrow 150. As shown in FIG. 13, the surgeon may continue to drive the wire 76 until the proximal tip 94 extends outwardly from the medial side of the metatarsal bone 18.

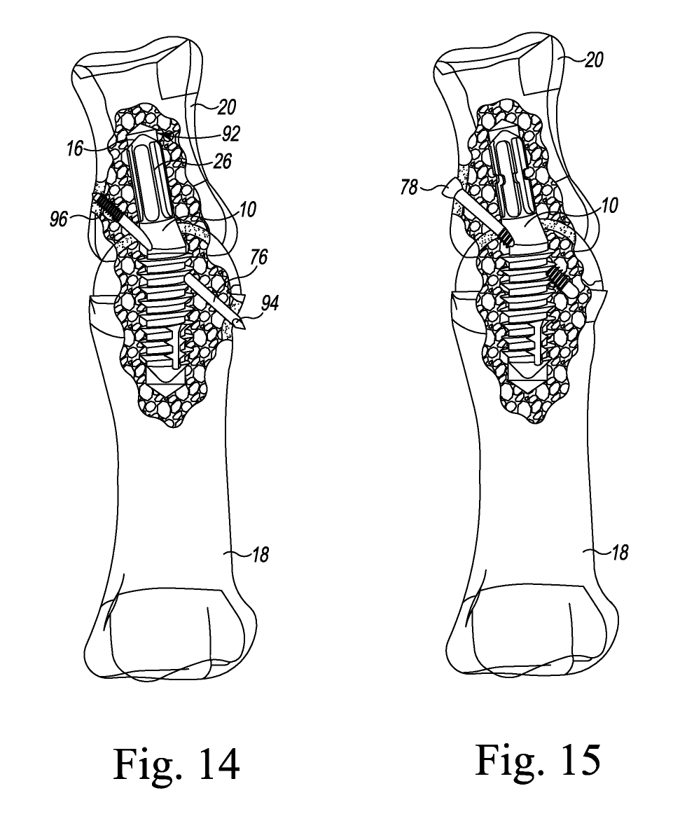

As shown in block 210 of the method of FIG. 9, the surgeon may secure the surgically-prepared proximal phalange 20 to the prosthetic component 10. To do so, surgeon may align the proximal opening of the bore 92 with the distal end 16 of the component 10. The surgeon may then drive the phalange 20 onto the distal body section 26 of the component. As the phalange 20 is advanced over the distal body section 26, the splines 46 engage the bone surrounding the bore 92 to create a press fit between the distal body section 26 and the phalange 20, thereby securing the component 10 to the bone. As shown in FIG. 14, a valgus angle of greater than 0 degrees is defined between the phalange 20 and the metatarsal bone 18 when viewed in the traverse plane of the patient's body. As described above, a dorsiflexion angle of greater than 0 degrees is defined between the phalange 20 and the metatarsal bone 18 when viewed in the sagittal plane of the patient's body.

When the phalange 20 is seated on the distal body section 26 of the component, the surgeon may advance to block 212. In block 212, the surgeon drives the Kirschner wire 76 into the phalange 20. To do so, the surgeon may drive the pointed tip 94 to advance the wire 76 distally through the passageway 70 of the component 10 and into contact with the phalange 20. The surgeon may continue tapping the wire 76 along this trajectory until positioned as shown in FIG. 14.

As shown in block 214 of the method of FIG. 6, the surgeon may then determine whether to use a bone screw 78 to provide additional fixation and some measure of compression at the joint interface. If the surgeon decides to use a bone screw, the method advances to block 216. In block 216, the surgeon advances the bone screw 78 into the phalange 20 and metatarsal bone 18, as shown in FIG. 15. It should be appreciated that the surgeon may use the wire 76 is a guide for the implantation of the bone screw 78. When the bone screw 78 is properly positioned, the surgeon may remove the wire 76. Alternatively, the surgeon may first remove the wire 76 prior to implanting the bone screw 78. It should be appreciated that in some embodiments the passageway 70 may be threaded at its proximal end to engage the threads of the bone screw 78. With the screw 78 positioned as shown in FIG. 15, the surgeon may then close the wound.

Returning to block 214, if the surgeon decides not to use a bone screw, the method advances to block 218. In block 218, the surgeon clips the ends of the Kirschner wire 76 close to the bone. The wire 76 is then left with the component 10 embedded in the patient's bones. The surgeon may then close the wound.

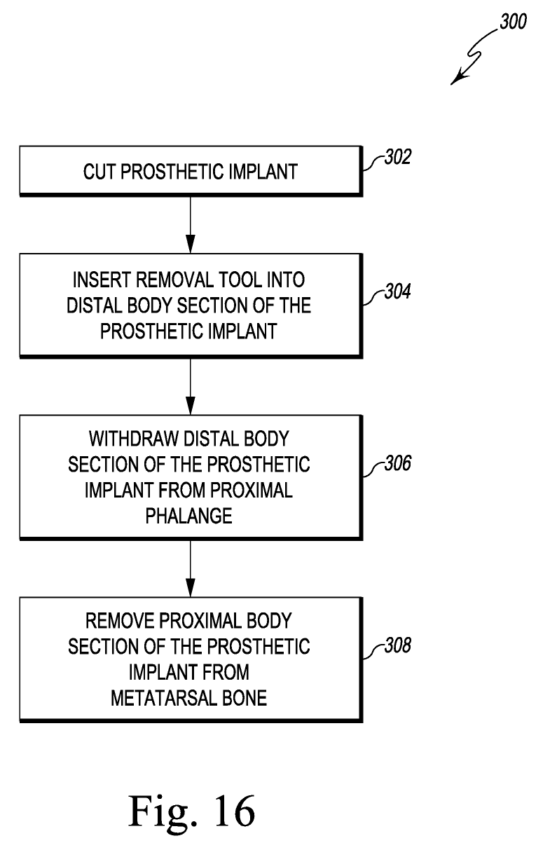

Referring now to FIG. 16, an exemplary method 300 removing an implanted prosthetic component 10 from a patient's joint is shown. The method 300 begins with the surgeon opening the tissue surrounding the joint area to permit access to the joint. In block 302, the surgeon may use a saw or other cutting tool to cut the component 10 into the proximal and distal sections. Specifically, the surgeon may advance the cutting tool into contact with the central body section 30 and cut through that body section to bisect the component 10.

The method 300 may then advance to block 304. In block 304, surgeon may insert a removal tool having a threaded distal end into the distal body section 26 of the component 10. With the removal tool engaged with the distal body section 26, the surgeon may, in block 306, pull the distal body section 26 in a proximal direction to withdraw the section 26 from the proximal phalange 20.

To remove the proximal body section 22 from the metatarsal bone 18 in block 308, the surgeon may thread the removal tool into the proximal body section 22. The surgeon may continue rotating the removal tool after it is bottomed on the body section to cause the proximal body section 22 to unscrew from the metatarsal bone 18. The surgeon may then proceed with further steps to complete the surgical procedure.

It should be appreciated that the concept of a single, monolithic prosthetic component configured for insertion into the bones of a joint may also be used in a hand or foot proximal interphalangeal joint fusion procedure.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A prosthetic component for use in a metatarso-phalangeal joint fusion procedure for a patient, comprising:
a single monolithic body including a first end sized to be positioned in a surgically-prepared bore defined in a proximal phalange of the patient's foot and a second end sized to be positioned in a surgically-prepared bore defined in a distal end of a metatarsal bone of the patient's foot, wherein the first end is laterally offset from the second end when the single monolithic body is viewed in a first plane.

2. The prosthetic component of claim 1, wherein the second end of the single monolithic body includes a plurality of threads configured to be threaded into the surgically-prepared bore of the distal end of the metatarsal bone of the patient's foot.

3. The prosthetic component of claim 1, wherein:
a first generally central axis extends through the first end of the single monolithic body,
a second generally central axis extends through the second end of the single monolithic body, and
an angle is defined between the first generally central axis and the second generally central axis when the single monolithic body is viewed in the first plane, the angle having a magnitude greater than 0 degrees.

4. The prosthetic component of claim 3, wherein the magnitude is less than or equal to 20 degrees.

5. The prosthetic component of claim 3, wherein the magnitude is equal to one of 5 degrees, 10 degrees, and 20 degrees.

6. The prosthetic component of claim 3, wherein a second angle is defined between the first generally central axis and the second generally central axis when the single monolithic body is viewed in a second plane extending orthogonal to the first plane, the second angle having a magnitude greater than 0 degrees.

7. The prosthetic component of claim 6, wherein the magnitude of the second angle is less than or equal to 35 degrees.

8. The prosthetic component of claim 6, wherein the magnitude of the second angle is equal to 20 degrees.

9. The prosthetic component of claim 6, wherein the first plane is a transverse plane and the second plane is a sagittal plane.

10. The prosthetic component of claim 6, wherein the single monolithic body includes:
a first body section including the first end and extending along the first generally central axis,
a second body section including the second end and extending along the second generally central axis,
a central body section extending along a third generally central axis from a first section end connected to the first body section to a second section end connected to the second body section, and
a third angle is defined between the first generally central axis and the third generally central axis when the single monolithic body is viewed in the first plane, the third angle having a magnitude greater than 0 degrees.

11. The prosthetic component of claim 1, wherein a spline extends along the first end of the single monolithic body, the spline being configured to engage bone surrounding the surgically-prepared bore defined in the proximal phalange of the patient's foot.

12. The prosthetic component of claim 11, wherein the spline includes a plurality of splines arranged around an outer circumference of the first end of the single monolithic body.

13. The prosthetic component of claim 1, wherein the single monolithic body has a passageway defined therein that is sized to receive a fastener.

14. The prosthetic component of claim 13, wherein the passageway is threaded at least one end.

15. A surgical kit comprising the prosthetic component of claim 1 and further comprising a surgical tool including an offset corresponding to the offset of the first end relative to the second end.

16. A prosthetic component for use in a metatarso-phalangeal joint fusion procedure for a patient, comprising:
a single monolithic body including (i) a distal body section that is sized to be positioned in a surgically-prepared bore defined in a proximal phalange of the patient's foot, and (ii) a proximal body section that is configured to be threaded into a surgically-prepared bore defined in a distal end of a metatarsal bone of the patient's foot,
wherein when the single monolithic body is viewed in a first plane, an angle is defined between a first generally central axis extending along the distal body section and a second generally central axis extending along the proximal body section, the angle having a magnitude greater than 0 degrees, and
wherein the distal body section is offset from the proximal body section when the single monolithic body is viewed in the first plane.

17. The prosthetic component of claim 16, wherein a second angle is defined between the first generally central axis and the second generally central axis when the single monolithic body is viewed in a second plane extending orthogonal to the first plane, the second angle having a magnitude greater than 0 degrees.

18. The prosthetic component of claim 16, wherein a spline extends along the distal body section, the spline being configured to engage bone surrounding the surgically-prepared bore defined in the proximal phalange of the patient's foot.

19. A method of performing a procedure on a patient, the method comprising:
preparing a proximal phalange of the patient's foot to receive a prosthetic component,
preparing a distal end of a metatarsal bone of the patient's foot to receive the prosthetic component,
selecting the prosthetic component including a first end and a plurality of threads on a second end opposite the first end,
threading the second end of the prosthetic component into the distal end of the metatarsal bone of the patient's foot,
inserting a fastener through a passageway defined in the prosthetic component, and
engaging the first end of the prosthetic component with the proximal phalange of the patient's foot after inserting the fastener.

20. The method of claim 19, wherein the fastener includes a Kirschner wire.
